(19) United States
(12) Patent Application Publication
(10) Pub. No.: US 2021/0010986 A1
(43) Pub. Date: Jan. 14, 2021

(54) PAPERLESS RECORDING PASTEURIZATION CONTROL SYSTEM

(71) Applicant: Anderson-Negele, Fultonville, NY (US)

(72) Inventors: Vikram A. Bose-Mullick, Fonda, NY (US); Gary Ratajczak, Broadalbin, NY (US); Richard J. Bond, Pattersonville, NY (US)

(21) Appl. No.: 17/037,448

(22) Filed: Sep. 29, 2020

Related U.S. Application Data
(63) Continuation of application No. 15/834,571, filed on Dec. 7, 2017.
(60) Provisional application No. 62/430,940, filed on Dec. 7, 2016.

(51) Int. Cl. G01N 33/04; A23L 3/00; G01F 1/688

(52) U.S. Cl. CPC G01N 33/04; G01F 1/688; A23L 3/003

(57) ABSTRACT

A pasteurization system includes at least a temperature sensor, a control signal generator, and a processor structured to record a stream of temperatures as well as their associated times of recordation, and to cause the control signal generator to generate a control signal based on the recorded temperatures.

### PRIORITY

This application is a continuation of co-pending U.S. non-provisional patent application Ser. No. 15/834,571, titled “PAPERLESS RECORDING PASTEURIZATION CONTROL SYSTEM,” filed Dec. 7, 2017, which is a non-provisional of and claims benefit from U.S. provisional patent application No. 62/430,940, titled “SYSTEMS AND METHODS FOR A PAPERLESS RECORDING CONTROL SYSTEM, filed Dec. 7, 2016, the disclosures of both of which are incorporated herein by reference in their entirety.

### FIELD OF THE INVENTION

This disclosure is directed to a pasteurization system such as may be used in the food and beverage industry, and in particular to pasteurizing dairy products.

### BACKGROUND

Many different types of products are pasteurized to reduce or to eliminate microorganisms. During a pasteurization process, a product is heated for a sufficient amount of time and at a sufficient temperature to kill all or substantially all of the microorganisms present in or on the product. The Food and Drug Administration (FDA) and other regulating entities have established standards for the pasteurization of specific products, such as for dairy products. These standards include the recommended minimum temperature to which the product should be heated and the recommend minimum time during which the product should be at or above the minimum temperature. If a product is not heated for a sufficient amount of time and/or at a sufficient temperature, serious consequences can result. The survival of microorganisms intended to be killed in the pasteurization process can create a health risk for the consumer and/or an economic loss for the producer.

Assuring the safety of food, beverage and dairy products while maintaining quality and increasing the shelf life is a significant challenge for the food industry. Originally, a batch process was relied upon to pasteurize dairy products, but this method has largely been replaced by continuous processes, which are more efficient and result in higher quality products. Several continuous pasteurization processes are available; for example, High-Temperature, Short-Time (HTST) pasteurization, Higher-Heat, Shorter-Time (HHST) pasteurization, or Ultra-High Temperature (UHT) pasteurization. The FDA specifies the range of acceptable process conditions for such pasteurization processes.

A dairy product processing plant may include multiple pasteurization systems. Each system is controlled by a separate controller and has its own data recorder, typically configured as a Safety Thermal Limit Recorder (STLR). The control set points for each system may be adjusted independently for proper pasteurization of the particular type of product processed at any given time, such as skim milk, whole milk, yogurt, eggs, etc.

In a typical known pasteurization system, temperature and flow rate of the heated food substance are measured by a thermometer device and flow metering device and that data is recorded by a circular chart recorder. The system includes a singular, fixed set point for each of the temperature and the flow rate to determine whether the food substance is pasteurized, which is recorded by the respective data record. If the food substance does not meet either of these set points, the food substance is returned to a holding tank to again flow through the pasteurization system again. However, the food substance may have already been adequately pasteurized despite not meeting either of the set points. Re-pasteurization of a food substance that has been adequately pasteurized may result in lower quality of the food substance, as well as create inefficiency in the overall pasteurization process by causing a pasteurized food substance to flow back through the pasteurization system.

Embodiments of the disclosure address these and other deficiencies in the prior art.

### BRIEF DESCRIPTION OF THE DRAWINGS

Aspects, features and advantages of embodiments of the present disclosure will become apparent from the following description of embodiments in reference to the appended drawings in which:

FIG. 1 is a block diagram of a paperless recording control system according to embodiments of the disclosure.

FIG. 2 is a more detailed block diagram of the paperless recording control system of FIG. 1.

FIG. 3 is a block diagram of a control environment in conjunction with the paperless recording control system of FIG. 1.

FIG. 4 is an example of a block diagram depicting the paperless recording control system of FIG. 1 in a pasteurization system.

FIG. 5 is a flow chart illustrating a process for pasteurizing a food substance in the pasteurization system of FIG. 4.

FIG. 6 is another example of a block diagram depicting the paperless recording control system of FIG. 1 in another pasteurization system.

FIG. 7 is an example circuit board assembly of a paperless recording control system.

FIGS. 8A and 8B are examples of circuit boards that may be utilized by the example circuit board assembly of FIG. 7.

FIG. 9 is an illustrative depiction of a front view of an enclosure for the paperless recording control system.

FIG. 10 is an illustrative depiction of a door panel of the enclosure of FIG. 9.

FIG. 11 is an illustrative depiction of a mounting interface panel of the enclosure of FIG. 9.

FIG. 12 is an illustrative depiction of an assembly door panel with a mounting interface panel of FIG. 11.

FIG. 13 is an illustrative depiction of an exterior view of mounting interface panel of FIG. 11 coupled with a plastic tub.

FIG. 14 is an illustrative depiction of an interior view of the enclosure of FIG. 9.

FIG. 15 is an illustrative depiction of an interior view of the enclosure of FIG. 9 having a paperless recording control system disposed therein.

FIG. 16 is an illustrative view of the card assembly depicted in FIG. 15.

FIG. 17 is an illustrative exploded view of an example pasteurization control interface (PCI) card assembly.

FIG. 18 is an illustrative view of an example card guide plate.

FIG. 19 is an illustrative view of an example circuit board assembly utilizing card guide plate of FIG. 18.

FIG. 20 is an illustrative view of an example card covering plate and a programming covering plate.

FIG. 21 is an illustrative component view of card covering plate and programming covering plate.

FIG. 22 is an internal view of an enclosure having a paperless recording control system with card covering plate and programming covering plate of FIG. 21.

FIG. 23 is another internal view of the enclosure depicted in FIG. 21.

FIG. 24 is a view of an example power supply assembly.

FIG. 25 is a view of an example power supply assembly covering plate.

FIG. 26 is a view of an enclosure for a paperless recording assembly integrated with a panel.

### DESCRIPTION

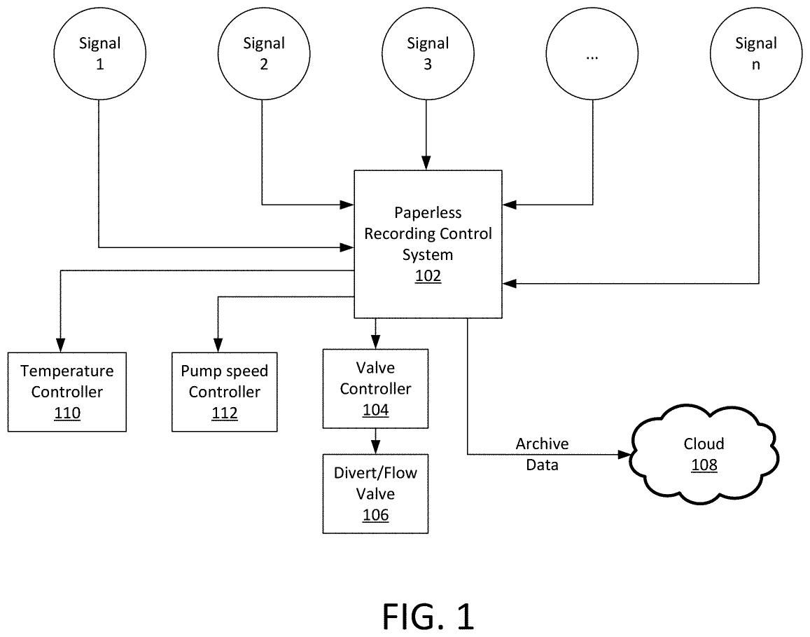

FIG. 1 depicts a control environment in accordance with various embodiments of the present disclosure. As depicted, the control environment includes a paperless recording control system 102. Paperless recording control system 102 can be configured to take as input, or receive, any number of signals, represented here as signals 1-n. In embodiments, these signals can be received from any suitable source, such as, for example, one or more sensor assemblies. Such sensor assemblies can include, but are not limited to, temperature sensors, flow sensors, pressure sensors, turbidity level sensors, a digital reference thermometer (such as the DART thermometer available from Anderson Instrument Co. Inc. of Fultonville, N.Y. 12072), or any other analytical sensor. In a particular embodiment, the sensors can include any sensors utilized in a pasteurization process.

The paperless recording control system 102 can be configured to process these signals to control a process, or aspects or sub-processes thereof, such as, for example, a pasteurization process. In embodiments, this control could be in accordance with established control systems methodologies, such as, for example, proportional-integral-derivative (PID) control methodology or any similar control algorithm. In such embodiments, paperless recording control system can be configured to output signals to the control mechanisms associated with the above described sensors, such as, for example, thermal control mechanisms, flow control mechanisms, pressure control mechanisms, etc.

In addition to controlling such a process, or sub-process, paperless recording control system 102 can, in some embodiments, also be configured to determine a point at which the prescribed process, or sub-process, has been successfully accomplished (e.g., when a product could legally be considered to have been pasteurized). In, such embodiments, the paperless recording control system 102 can be configured to output a signal indicating that the process has completed successfully. Such a signal could be, for example, a valve control signal that is configured to cause a valve of a pasteurization process to open to allow product undergoing pasteurization to exit the pasteurization process (e.g., to enter a holding tank). Such a signal is depicted by the arrow exiting the paperless recording control system 102 and entering valve controller 104. In the depicted embodiment, valve controller 104 can receive the signal and, in response cause a divert/flow valve 106 of the pasteurization process to open, thus allowing pasteurized product to exit the pasteurization process. In embodiments, this signal can be a low voltage DC signal (e.g., 40 volts). Other output signals may include a temperature control signal this is configured to cause a pasteurization tube to be heated to a particular system and a pump-speed control signal that is configured to control the speed of a pump to pump a food substance through the pasteurization process.

Paperless recording control system 102 can also be configured to record values of the incoming signals for data recording and display purposes. Such data recording purposes could include, for example, data sufficient to validate that a pasteurization process was successfully completed. In embodiments, paperless recording control system 102 could have a local archive of these recorded values which can be verified via a human machine interface (HMI) provided by the paperless recording control system 102. In embodiments, the local archive can include sufficient storage to preserve two years of data produced and/or received by the paperless recording control system. In addition to the local archive, the paperless recording control system 102 can also be connected with one or more external repositories, such as, for example cloud 108. As used herein, cloud 108 can represent and include any number of servers, including fractional server usage associated with, for example, a data center. Archiving can be performed periodically (every day, as space is need in the local archive, etc.) or on demand (e.g., based on input from a user of the paperless recording control system 102. In addition to, or in place of, cloud 108, paperless recording control system can also include removable media (e.g., a memory card, thumb drive, etc.) that can be removed for archival purposes. In such embodiments, the local archive of the paperless recording control system 102 can include a redundant copy of the data that is included on the memory card or thumb drive. In addition, the data can be encrypted in any number of ways to prevent tampering with the data. One such mechanism could include encryption of the data via a trusted platform module (TPM), or similar mechanism, of the paperless recording control system 102 such that the data is effectively sealed to the paperless recording control system 102. It will be appreciated that encryption via a TPM is just one possible mechanism for encrypting the data to prevent tampering of the data and any encryption mechanism (e.g., public/private key encryption) can be utilized to enable viewing the data without providing the ability to modify the data to maintain data integrity. Additional aspects of the paperless recording control system 102 will be readily apparent throughout the disclosure provided for herein.

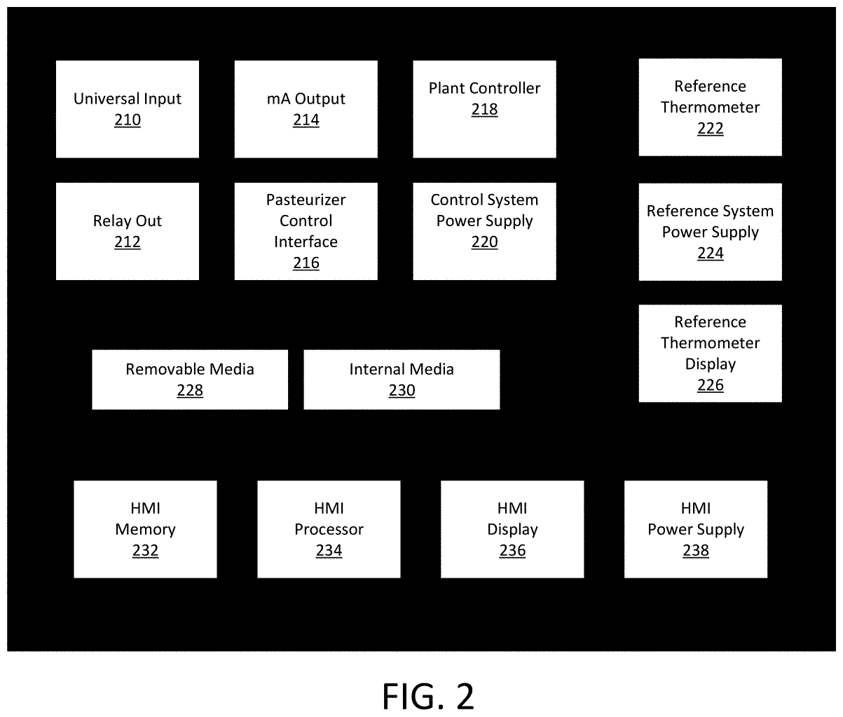

FIG. 2 depicts a more detailed view of paperless recording control system 102 of FIG. 1. As depicted, paperless recording control system 102 can include a control system 202, a reference system 204, storage 206, and HMI system 208. Each of these systems will be discussed in turn.

Control system 202 includes a number of illustrative components. As depicted, control system 202 includes a universal input 210, a relay out 212, a milliamp (mA) output 214, a pasteurizer control interface 216, and a control system power supply 220. Universal input 210 represents a component that can be utilized to receive one or more signals such as those described in detail in reference to FIG. 1 (e.g., signals 1-n depicted in FIG. 1, or any other signals described herein). In embodiments, any number of universal inputs 210 can be included within control system 202 to receive any number of signals. The number of universal inputs 210 could be dictated, for example, by the particular application of paperless recording control system 102.

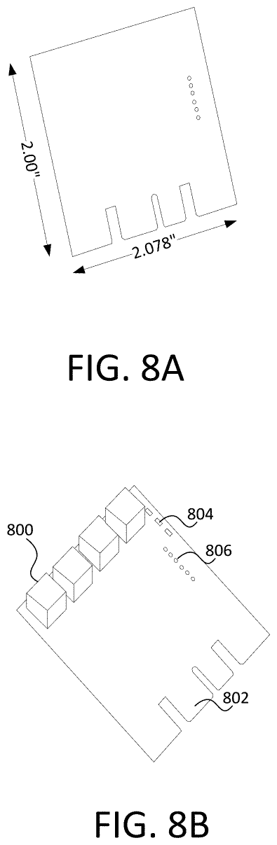

Relay out 212 can be configured to operate in a similar manner to any other relay component currently utilized. An example of such a component is depicted in FIG. 8.

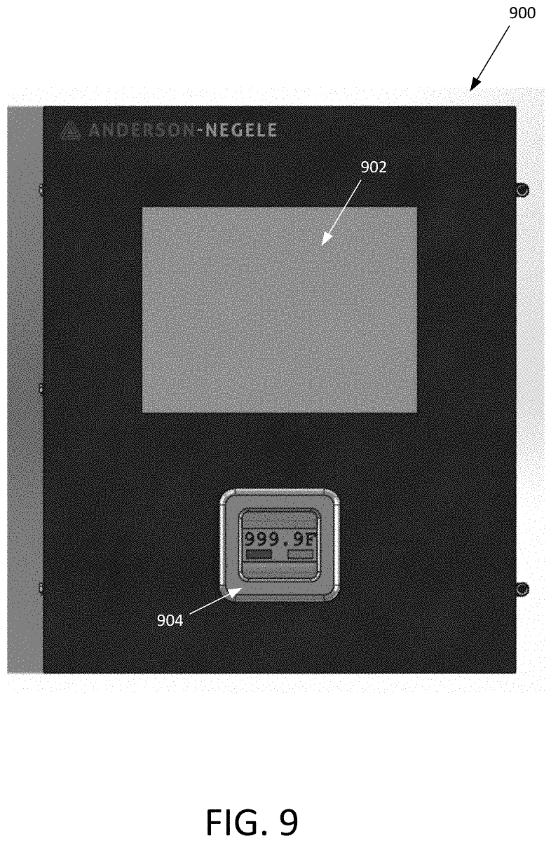

The mA output 214 component represents a component configured to provide a measurement signal based on a measurement of an associated signal (e.g., any of signals 1-n of FIG. 1) where an adjustment in current output by mA output 214 reflects an adjustment in the measurement of the associated signal. In embodiments, the measurement signal can range from 4 to 20 mA where any measurement of the associated signal maps to a specific current output by mA output 214. An example of such a component is depicted in FIG. 9.

The pasteurizer control interface (PCI) 216 represents a component that outputs one or more control signals that are configured to control aspects of a pasteurization process. Such signals could include, for example, the signal provided to valve controller 104 of FIG. 1, discussed above.

The plant controller 218 represents a master controller of control system 202. As such plant controller 218 can power cycle any of the other depicted components. In addition, plant controller 218 can take the values of the input signals and can carry out proportional-integral-derivative (PID) control system processes, or other similar control processes, to manage the process being carried out by the paperless recording control system.

It will be appreciated that the depicted components merely represent an illustrative configuration of control system 202 and that more or fewer components can be included without departing from the scope of this disclosure. For example, as mentioned previously, an encryption component, such as a hardware encryption component (e.g., TPM), could also be included to encrypt data being output by the control system 202 in order to maintain integrity of the data. In addition, each of these components can be implemented in any number of ways, such as, for example, via hardware, software, or any combination thereof. In some embodiments, control system 202 could be implemented via the backplane configuration discussed in detail below. It should be appreciated though, that the backplane configuration discussed herein is merely one possible configuration that can be utilized to implement the functionality provided by control system 202.

It should be appreciated that, in embodiments, control system 202 may need to be fully functional while the associated process is being carried out (e.g., a pasteurization process). In such embodiments, control system 202 represents a hard real-time system where any missed responses (e.g., deadlines) represent total system failure. In such embodiments, it can be desirable to isolate HMI system 208 and control system 202 (as depicted). This can enable the HMI functionality of HMI system 208 to be dissociated from the control and data gathering functionality of control system 202. By dissociating HMI system 208 from control system 202, a failure with the HMI system 208 will not impact the functionality of control system 202, thereby enabling continued recording of data and control of the associated process, even when the HMI system has encountered an issue.

Paperless recording control system also includes a reference system 204. Reference system 204 can include, for example, a reference thermometer (e.g., the previously mentioned DART thermometer) that is configured to measure a current temperature, a reference system power supply 224 that is configured to provide an independent source of power to the reference system 204, and a reference thermometer display 226 that can be configured to display a measurement of the reference thermometer to a user of the paperless recording control system 102. While depicted as a reference thermometer, it will be appreciated that any other reference sensors could be utilized without departing from the scope of this disclosure.

As depicted, reference system 204 can be completely isolated and independent from the other functionality of paperless recording control system 102. This isolation can maintain the integrity of reference system 204. In other embodiments, the reference system 204 can be external to the paperless recording control system 102. In either of these embodiments, reference system 204 can be configured to output a reference signal that indicates a current measurement of the reference system 204. Such a signal can be utilized for performing an automated cut-in/cut-out process. Under the current state of the art, an operator of a pasteurization process must manually record the temperature of the reference thermometer to be compared with a temperature of a thermometer of the pasteurization system. If the difference in temperature between the two readings is greater than a certain threshold (e.g., 1 degree), then the pasteurization system is not operating properly and corrective measures may need to be taken. By providing a digital signal from the reference thermometer, regardless of whether the reference thermometer is internal or external to the paperless recording control system 102, then the digital signal can be utilized by the paperless recording control system 102 to perform an automated cut-in/cut-out process and remove any chance of operator error. This can be accomplished, for example, by providing a button via the HMI that is configured to have such a process carried out. In other embodiments, the process can be carried out automatically (e.g., when a new operator logs into the paperless recording control system 102). This automation can be configured similarly to any of the other settings of the paperless recording control system 102 discussed in greater below.

Paperless recording control system 102 also includes storage 206. Storage 206 can include removable media (e.g., a thumb-drive, secure digital (SD) card, etc.) as well as internal media 230 that can be utilized to store a local archive of data. In embodiments, storage 206 can be written to by control system 202 and can be accessed by HMI system 208. In some embodiments, storage 206 could be integrated with HMI system 208. HMI system 208 is discussed in greater detail below. In embodiments, the removable media 228 can be removed by an operator to be copied to an external archive. In such embodiments, upon re-inserting removable media 228, removable media can be updated with any new data that has been stored on internal media 230. In some embodiments, to ensure the integrity of the data on removable media 228, the internal archive of internal media 230 can be copied over anything already stored on removable media 228, to ensure an exact copy of the local archive is also stored on removable media 228. As discussed previously, the data stored on removable media 228 and internal media 230 can be encrypted. In some embodiments, internal media 230 can have sufficient capacity to store a great deal of data (e.g., 2 years of operating data) in the local archive.

Finally, paperless recording control system 102 includes an HMI system 208. As depicted, HMI system 208 is completely isolated from both the control system 202 and the reference system 204. As such, HMI System 208 includes independent HMI memory 232, an independent HMI processor 234, an independent HMI display 236 (e.g., a touchscreen), and an independent HMI power supply 238. In embodiments, HMI memory 232 can include software, which when executed by HMI processor 234 cause the HMI system to carry out any of the HMI processes described and/or depicted herein. In some embodiments, HMI system can also include an embedded web service (EWS) to enable remote access to the paperless recording control system 102. Such remote access can enable a user to remotely adjust configuration parameters, such as those discussed in detail below, or to view any of the screens depicted herein. In some embodiments, the EWS can take the place of HMI display 236, or can be implemented in conjunction with HMI display 236.

While not depicted, it will also be appreciated that paperless recording control system can also include a transmitter/receiver that is configured for remote communication. This could be, for example any wired or wireless transmitter/receiver. Such a transmitter/receiver combination can utilize any communication mechanism including a local network, the internet, a cellular communication network, a personal area network (e.g., Zigbee) or any other suitable mechanism for communication. Such a transmitter/receiver could be integrated as part of HMI system 208 to ensure the transmitter/receiver is isolated from control system 202.

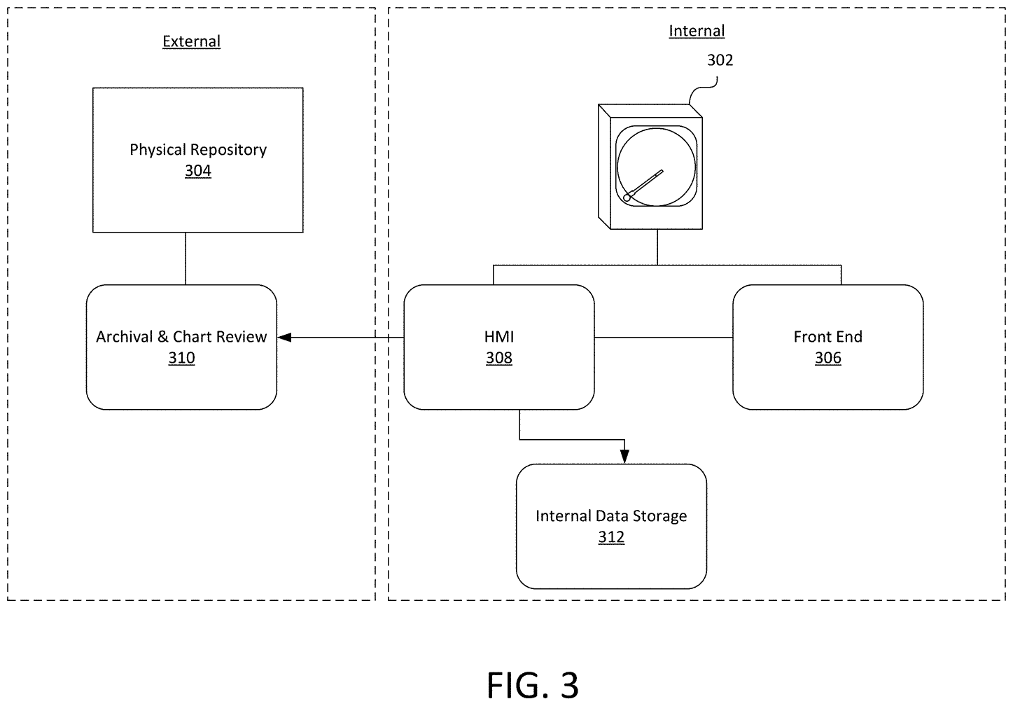

FIG. 3 is a depiction of a current state of the art control environment in conjunction with a novel paperless recording control system as described herein. Under the current state of the art, a control environment (e.g., a pasteurization control environment) includes a drag pen style circular chart recording controller 302, such as the AJ-300 or AV-9000 recording controller. A drag pen style circular chart recorder works by moving a piece of paper at a steady rate while a drag pen is positioned to record various sensor measurements on the paper. Each of these charts are then stored for review in a physical repository 304. Under the described paperless recording control system these components are replaced with a front end 306 (e.g. control system 202) and an HMI system 308 (e.g., HMI system 206 of FIG. 2). The HMI system 308 may include an internal data storage 312. The physical repository is also replaced by chart archival and storage for archival and chart review 310, which may be external to the paperless recorder.

The HMI 206 discussed elsewhere herein may be divided into a PC software component, which can interact with the HMI 206, and the HMI 206 itself. In other embodiments, all functionality accessible via the HMI can also be accessible via PC software, and vice versa.

In some embodiments, the PC software, or the HMI 206, can enable remote setting of various set points. For example, in some embodiments, the paperless recording control system can be configured to be pasteurizing eggs. When pasteurizing eggs, the temperature set point is set to various values throughout the process. In such an embodiment, remote setting of the temperature set points may be enabled for egg pasteurization only, and could be restricted for any other pasteurization process, such as dairy. In other embodiments, multiple flow set points can be defined, via the HMI or PC software, that correspond with various temperature set points.

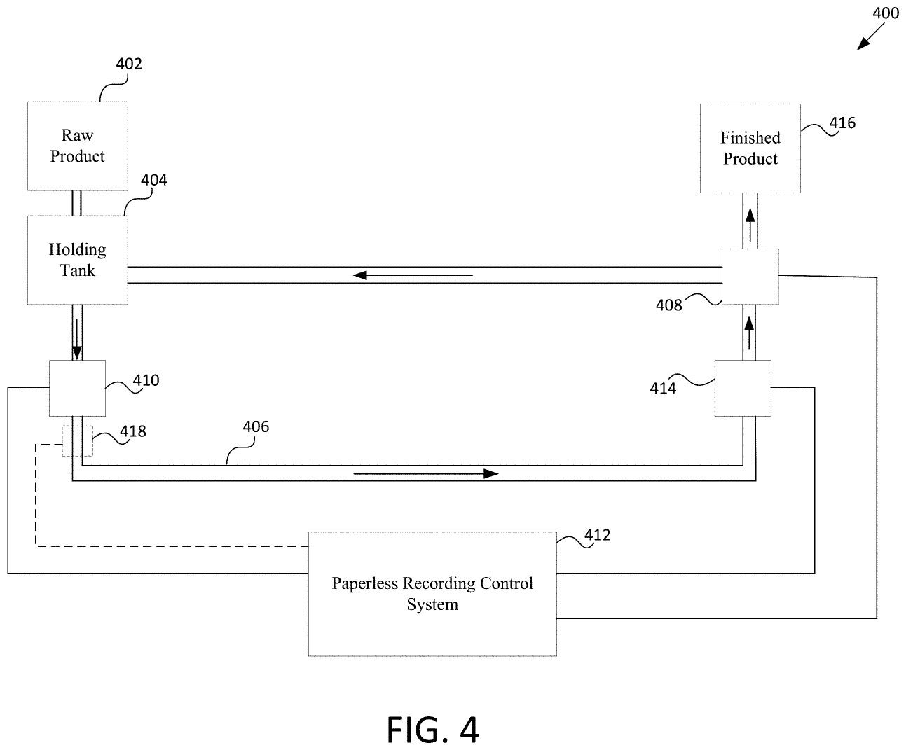

FIG. 4 depicts an example block diagram of a simplified pasteurization system 400 including the paperless recording control system of FIG. 1, according to some embodiments of the disclosure. The pasteurization system 400 of FIG. 4 may include additional components, not shown, such as heat exchangers, homogenizers, pressure switches, etc., as would be understood by one skilled in the art.

A food substance that is unpasteurized and held in a raw product tank 402 flows into a holding tank 404 prior to pasteurization. The food substance flows through a pasteurization tube 406, which may also be referred to as piping, to a valve 408. While in the pasteurization tube 406, the food substance is heated by a heating element (not shown). A flow sensor 410 measures the flow of the food substance as it enters or proximate to the entry of the pasteurization tube 406. An output from the flow sensor 410 is sent through either wireless or wired communication to a paperless recording control system 412, which may be a paperless recording control system 102, as discussed in detail above. A temperature sensor 414 measures the temperature of the food substance at or near an end of the pasteurization tube 406 and sends wirelessly or wired the measured temperature to the paperless recording control system 412.

In some embodiments, an optional second temperature sensor 418 may be included at the beginning or proximal portion of the pasteurization tube 406. The output of the second temperature sensor 418 is sent to the paperless recording control system 412 and may be used by the paperless recording control system 412 to predetermine a likelihood that a temperature taken by the temperature sensor 414 will indicate that the food substance is pasteurized or may be used for other determinations and calculations by the paperless recording control system 412.

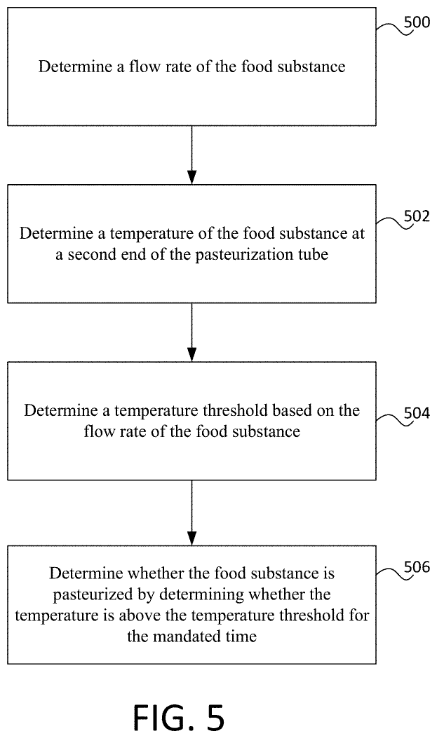

As discussed in more detail with respect to FIG. 5, the paperless recording control system 412 determines whether the food substance has been pasteurized based on a combination of both the flow rate of the food substance and the temperature of the food substance. If the food substance is pasteurized, the valve 408 directs the food substance to a finished product tank 416 (FIG. 4). If the paperless recording control system 412 determines that the food substance has not been pasteurized or if there is any other error, the paperless recording control system 412 sends a signal to the valve 408 to direct the food substance back to the holding tank 404 to flow through the pasteurization system 400 again.

FIG. 5 illustrates a process for determining whether the food substance has been adequately pasteurized through the pasteurization system 400. Known food pasteurization systems determine whether either of the flow rate or the temperature are outside a predetermined, fixed threshold, and if so, the system indicates that the food substance has not been adequately pasteurized and will return the food substance to the holding tank 404.

For example, if the temperature is lower than the set point, then the food substance would need to flow through the pasteurization system at a slower speed to be adequately pasteurized. However, the prior art pasteurization systems would determine the food substance was not pasteurized based on the temperature alone without looking at the flow rate of the food substance. If the food substance had flowed through the pasteurization tube at a slower flow rate, then the food substance was pasteurized but is sent back to the holding tank 404 to be pasteurized again. Further, if the food substance flowed through the pasteurization tube quicker than the set point, the pasteurization system would again determine that the food substance was not pasteurized, without taking into consideration the temperature. If the temperature was high enough, the food may have been pasteurized despite the high flow rate of the food substance.

Embodiments of the disclosure, however, determine a threshold temperature based on the flow rate and then determine whether the temperature is above that threshold. That is, the temperature set point is variable based on the flow rate of the food substance. As illustrated in FIG. 5, at operation 500, the paperless recording control system 412 receives and determines the flow rate of the food substance. At operation 502, the paperless recording control system 412 also receives and determines the temperature of the food substance. Based on the flow rate received, the paperless recording control system 412 determines an acceptable temperature threshold for the food substance in operation 504. If the temperature is greater than the acceptable temperature threshold for a mandated amount of time, then, at operation 506, the paperless recording control system 412 determines that the food substance is pasteurized and can move to the finished product tank 416. Otherwise the food substance is returned to the holding tank 404.

Using the process of FIG. 5, the paperless recording control system 412 is able to vary the required temperature set point for a particular food substance throughout an entire pasteurization process. That is, the temperature set point for pasteurization will vary as the flow of the food substance varies. For example, pasteurization may be achieved at a lower temperature when the food substance is flowing at a relatively slow rate. Conversely, relatively fast flow rates require an elevated temperature to achieve pasteurization. Embodiments of the disclosure include determining a one-to-one relationship for flow rate and temperature. That is, a minimum threshold temperature will vary based on different flow rates, and vice versa. Then, comparing a measured temperature for a food substance having a particular flow rate, to determine if the minimum temperature has been reached. If so, then the pasteurization has been achieved. If not, the food substance is returned to the holding tank 414 to be re-pasteurized.

In some embodiments, the paperless recording control system 412 may also determine the quality of the food substance after pasteurization. That is, the paperless recording control system 412 will determine two acceptable temperature thresholds for the food substance. The first acceptable temperature threshold is to determine whether the food substance has been pasteurized, as discussed in detail above. The second acceptable temperature threshold is a threshold larger than the first threshold and relates to the quality of the food substance. If the food substance is pasteurized at too high of a temperature, the food substance may not meet the quality standards desired. In such a situation, the valve 408 may have an additional outlet (not shown) that diverts the food substance to a tank to store the food substance that does not meet the quality standard. Similar to the first acceptable temperature threshold, the second acceptable temperature threshold is determined based on the flow rate of the food substance.

As mentioned above, the acceptable threshold temperatures are determined based on the flow rate of the food substance. That is, an acceptable temperature will vary with the flow rate of the food substance. The acceptable threshold is calculated based on the length of the pasteurization tube and the flow rate of the food substance through the pasteurization tube. The paperless recording control system 412 may store a length, diameter, or other quality of the pasteurization tube in memory and calculate an acceptable temperature threshold based on the received flow rate. In some embodiments, a look-up table may be stored in the memory which is determined based on the length of the pasteurization tube. Instead of performing a calculation each time a flow rate is received, the paperless recording control system 412 may use the look-up table to more quickly determine an acceptable temperature threshold. In other embodiments, a user may create the look up table through the HMI of the paperless recording control system 412. The user may enter a variety of acceptable flow rate and temperature set points to determine pasteurization of the food substance.

In some embodiments, a user may enter a food substance type within the HMI of the paperless recording control system 412. The paperless recording control system 412 includes a memory that has a database indicating a variety of food substances, such as milk, yogurt, egg mixtures, etc., that a user may select through the HMI of the paperless recording control system 412. Once selected, the paperless recording control system 412 determines the acceptable temperature threshold based on both the flow rate of the food substance as well as the type of food substance. That is, the memory may include a formula for each food substance type saved to calculate the acceptable threshold temperature based on the length of the pasteurization tube 406 and the flow rate of the food substance or the memory may include a look-up table for each of the food substance types to even more quickly determine the acceptable threshold temperature for the flow rate of that food substance.

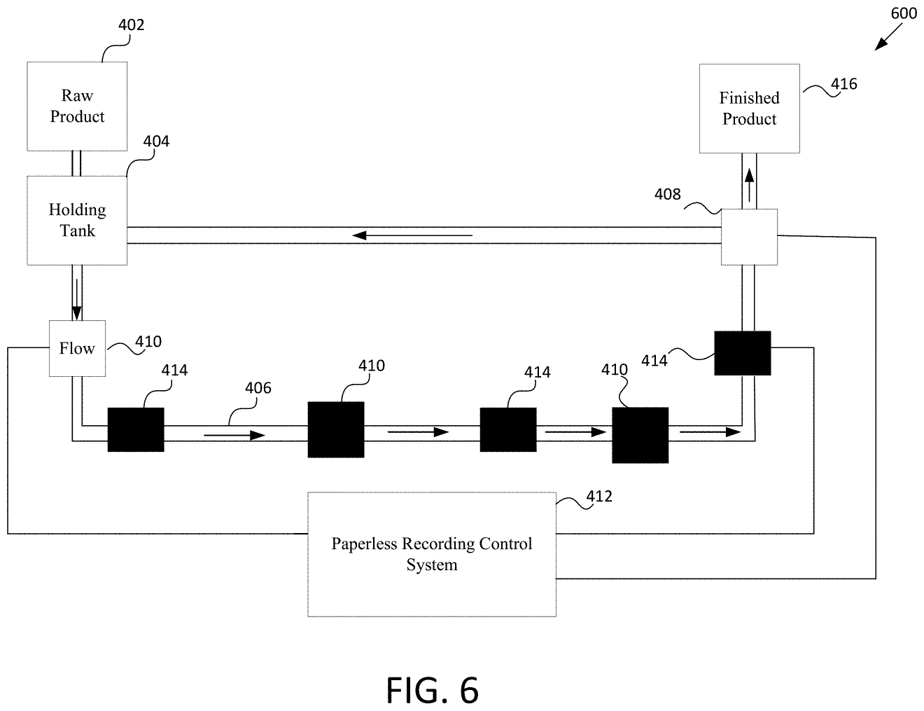

FIG. 6 depicts another example of a block diagram of a simplified pasteurization system 600 according to some embodiments of the disclosure. Similar to the pasteurization system 400 of FIG. 4, the pasteurization system 600 may include additional components, not shown, such as heat exchangers, homogenizers, pressure switches, etc., as would be understood by one skilled in the art.

The pasteurization system 600 includes components similar to those discussed above with respect to FIG. 4 and such components are not discussed here in further detail. In some pasteurization system, a food substance may have a variety of processes performed on it that may require the system 600 to include multiple sections of pasteurization tube 406. That is, the pasteurization system may have many flow sensors 410 and/or temperature sensors 414. That is, the paperless recording control system 412 may receive multiple flow rates or temperatures of the food substance throughout the process of pasteurization to confirm that food is adequately pasteurized and/or the quality of the food.

For example, the pasteurization system may have one flow sensor 410 and multiple temperature sensors 414. In other embodiments, there may be provided a temperature sensor 414 for every flow sensor 410, etc. As discussed above, the food substance type may be selected by a user and the paperless recording control system 412 may select the required temperatures throughout the process based on the flow rate and the food substance type, such as an egg mixture which may require multiple temperature points, as discussed above.

In some embodiments, as would be understood by one skilled in the art, an acceptable flow rate threshold may be determined based on the temperature rather than an acceptable temperature threshold determined based on the flow rate, as discussed above. In such embodiments, after determining the temperature of the food substance, the systems 400 and 600 may determine an acceptable flow rate for the determined temperature. If the flow rate is less than or equal to the acceptable flow rate threshold, then the pasteurization system determines the food has been pasteurized and the valve 408 diverts the food to a finished product tank 416.

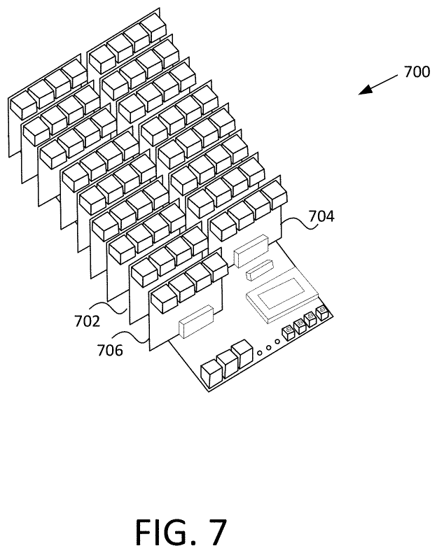

FIG. 7 depicts an example circuit board assembly 700 of a paperless recording control system, in accordance with various embodiments described herein. As can be seen, the circuit board assembly 700 depicted is in a backplane configuration, however, this configuration is not intended to be limiting of the possible configurations. It will be appreciated that other configurations could be readily ascertained by a person of ordinary skill in the art from the disclosure herein, and these other configurations are expressly contemplated for herein as being within the scope of this disclosure.

As depicted, the circuit board assembly 700 includes a total of nineteen circuit boards coupled together via a backplane circuit board. It will be appreciated that the nineteen circuit boards are merely meant to be illustrative in nature and that any number of circuit boards can be utilized without departing from the scope of this disclosure.

The backplane circuit board can provide for communication between any of the nineteen circuit boards depicted. As depicted, the nineteen circuit boards include a pasteurizer control interface 702, a dart card 704 (e.g., reference thermometer), and a plant controller card 706. The pasteurizer control interface 702 corresponds with the previously described pasteurizer control interface 216 of FIG. 2, the dart card 704 corresponds with the reference thermometer component 222 of FIG. 2, and the plant controller card corresponds with the plant controller 218 of FIG. 2. It will be appreciated that, in embodiments where a reference thermometer is not included as part of the backplane circuit board, the dart card 704 could be omitted. The remaining circuit boards could be comprised of circuit boards configured to comport with the previously described universal input component 210, relay out component 212, or mA output component 214, all of FIG. 2. The composition of these remaining circuit boards would be implementation specific and may vary from one implementation to another depending on the requirements associated with each implementation.

FIGS. 8A and 8B depict illustrative circuit boards that can be utilized as part of the backplane system depicted in FIG. 5. It will be appreciated that the dimensions depicted in FIG. 8A are meant to be illustrative in nature and are not intended to be limiting of this disclosure. Moving to FIG. 8B, the customer wiring terminals 800 provide for input to the card, while the card edge 802 provides for structural support and signal transfer between the card and the remainder of the respective paperless recording control system. While four customer wiring terminals 800 are depicted, it will be appreciated that this is merely for illustration and more or fewer customer wiring terminals could be included without departing from the scope of this disclosure. The four LEDs 804 can provide indicators of various states associated with the customer wiring terminals, or other states associated with the paperless recording control system. In addition, the programming connector 806 can provide access to implement or change one or more programming parameters associated with the circuit board.

FIG. 9 is an illustrative depiction of a front view of an example enclosure 900 for a paperless recording control system, in accordance with various embodiments of the present disclosure. As depicted, the example enclosure 900 includes a display screen 902 (e.g., a touch screen) and a reference thermometer display 904. Display screen 902 can correspond with HMI display 236 of FIG. 2 and reference thermometer display 904 can correspond with reference thermometer display 226 of FIG. 2. It will be appreciated that, in some embodiments, access to the HMI can be accomplished through remote access only (e.g., over the internet via an EWS). In such embodiments, display screen 902 can be omitted.

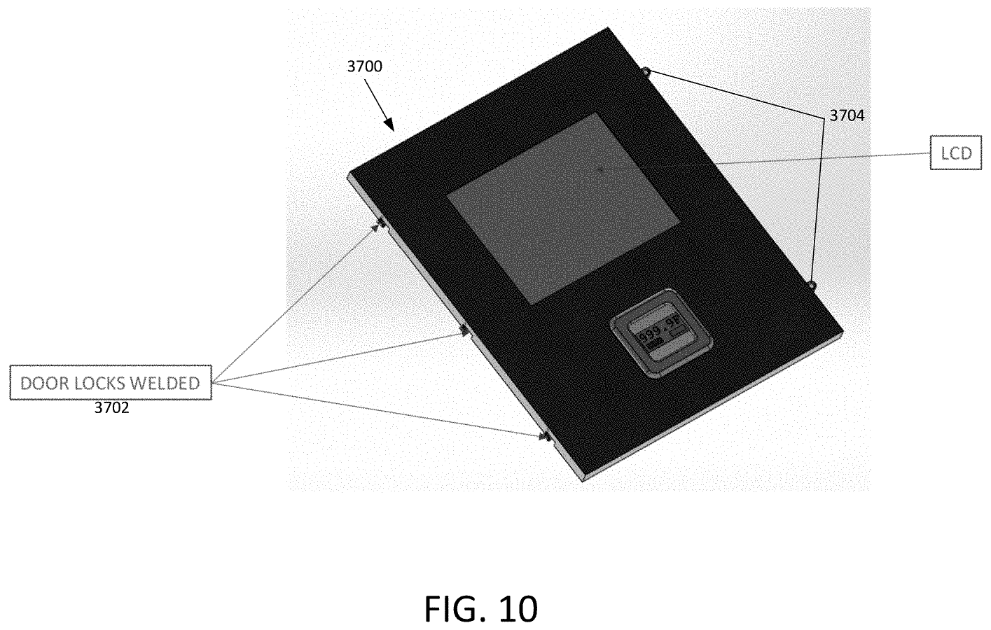

FIG. 10 is an illustrative depiction of a door panel 1000 of example enclosure 900 for a paperless recording control system, in accordance with various embodiments of the present disclosure. The door panel 1000 can be configured to be coupled with the enclosure 900 via door hinge welding locations 1002. In addition, the door panel 1000 can be securely closed utilizing door panel locking mechanism 1004.

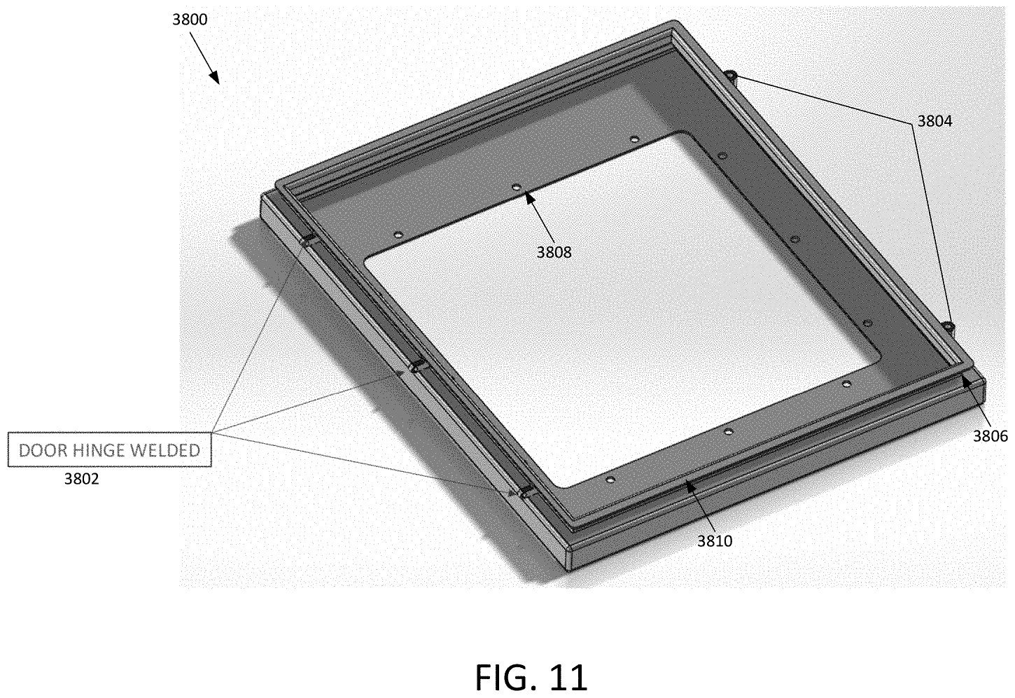

FIG. 11 is an illustrative depiction of a mounting interface panel 1100 of example enclosure 900 for a paperless recording control system, in accordance with various embodiments of the present disclosure. The mounting interface panel 1100 can be configured to have door panel 1000 coupled with the mounting interface panel 1100 via interface hinge welding locations 1102 (i.e., utilizing door hinge welding locations 1002). In addition, the door panel 1000 can be securely closed utilizing panel locking mechanism 1104, which can be configured to interface with door panel locking mechanism 1004. In embodiments, the enclosure may need to maintain a water-tight seal capable of withstanding a certain amount of water pressure (e.g., the water-proofing standards implemented by the National Electrical Manufacturers Association (NEMA)). In such embodiments, a seal/gasket may interface with mounting interface panel between sealing surface 1110 and door panel 1000. In order to help maintain the integrity of such a seal, mounting interface panel can include a liquid deflection channel 1106 that is configured to help redirect water away from the seal/gasket. It will be appreciated that such a liquid deflection channel could alternatively be integrated with the door in other embodiments. Mounting interface panel 1100 can also be configured to have a tub (e.g., the plastic tub discussed below) attached to a rear surface of the mounting interface panel 1100 via a suitable attachment mechanism (e.g., screw holes 1108). In embodiments where the enclosure needs to maintain a water-tight seal, such as that described above, a seal/gasket can also be disposed between the mounting interface panel 1100 and the attached tub.

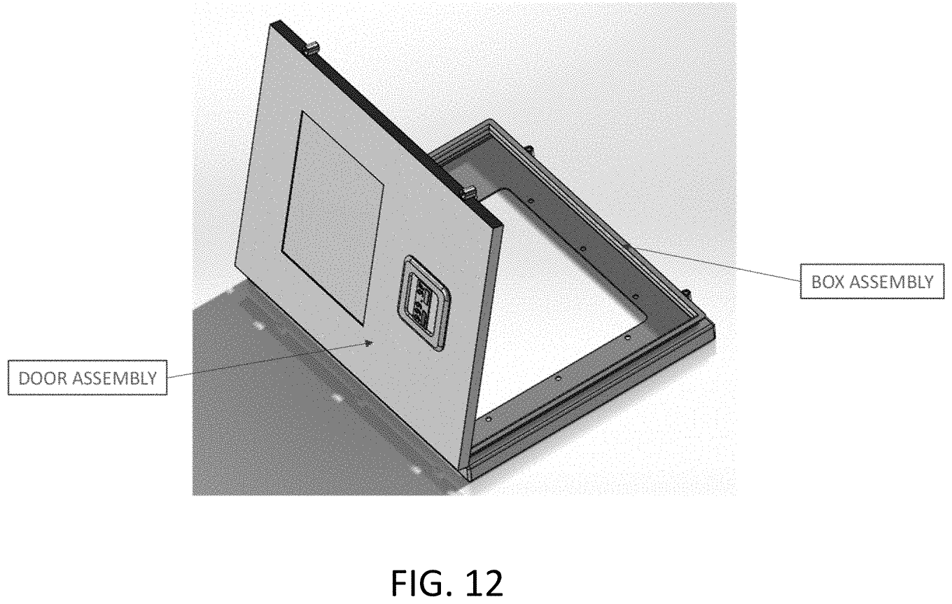

FIG. 12 is an illustrative depiction of an assembly of door panel 1000 with mounting interface panel 1100 (i.e., box assembly), in accordance with various embodiments of the present disclosure.

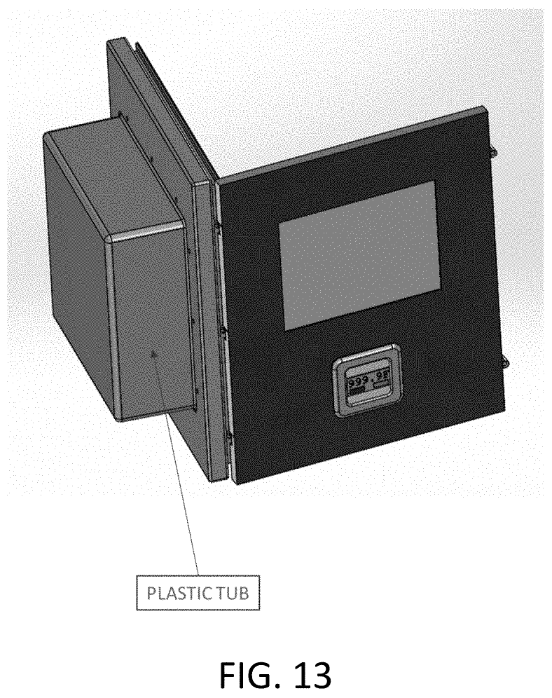

FIG. 13 is an illustrative depiction of an exterior view of mounting interface panel 1100 coupled with a plastic tub. While depicted as being a plastic tub, it will be appreciated that the tub, as well as the other components of the example enclosure, can be made from any suitable material without departing from the scope of this disclosure.

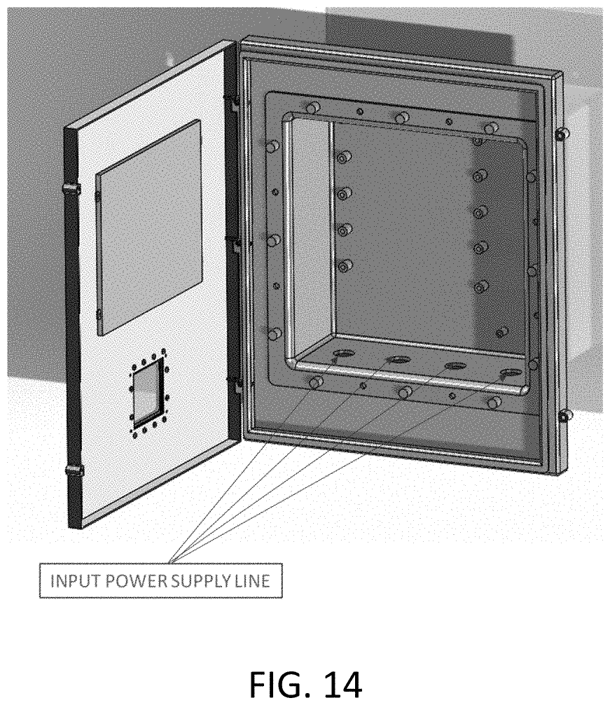

FIG. 14 is an illustrative depiction of an interior view of example enclosure 900, in accordance with various embodiments of the present disclosure. As can be seen, the interior of example enclosure includes a number of holes through which input power supply lines, along with sensor signal wiring, can be routed to be utilized by the paperless recording control system.

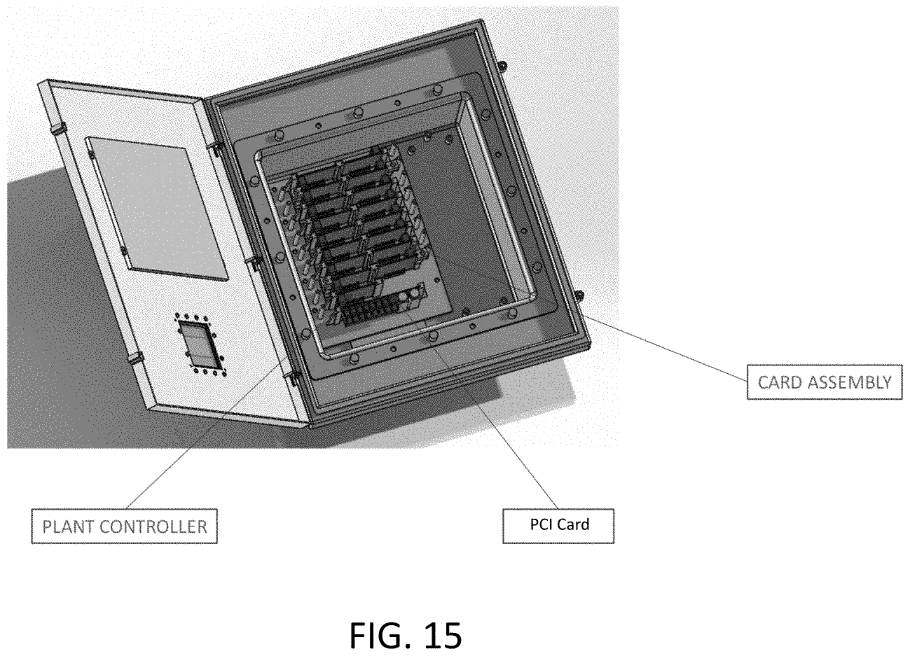

FIG. 15 is an illustrative depiction of an interior view of an example enclosure having a paperless recording control system disposed therein, in accordance with various embodiments of the present disclosure. As can be seen, the card assembly is installed into the plastic tub depicted by FIG. 13, above. It will be appreciated that cabling between the card assembly, also referred to herein as a circuit board assembly, and the HMI and reference thermometer displays to prevent obscuring the depicted embodiment. As depicted, the card assembly includes a pasteurization control interface card and a plant controller, along with any number of other cards, or circuit boards, described above.

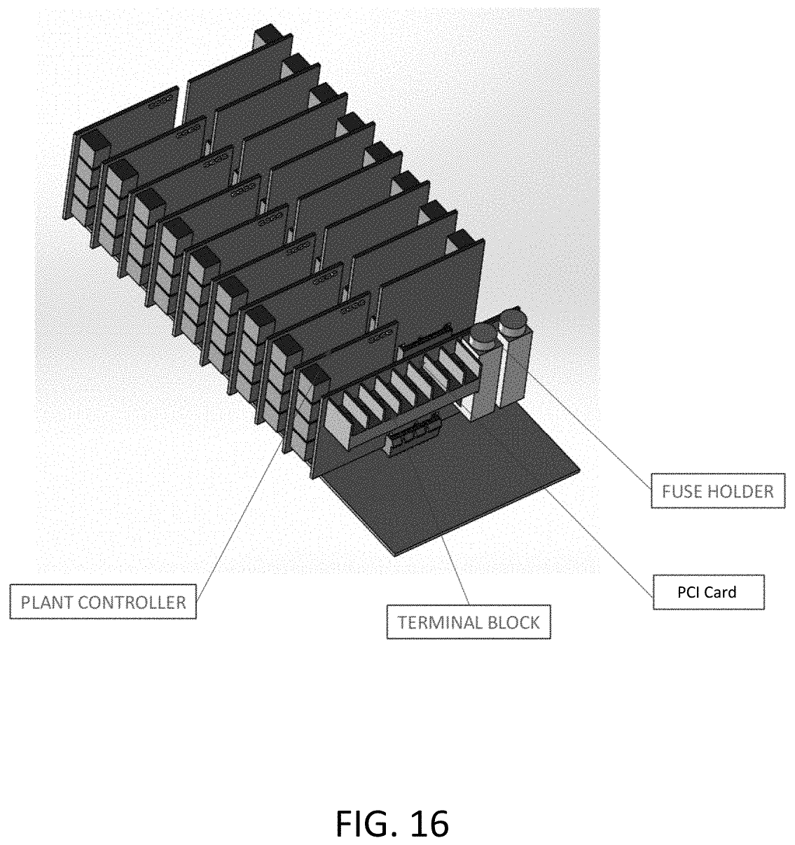

FIG. 16 is an illustrative view of the card assembly depicted in FIG. 15. As depicted, the card assembly includes a plant controller card and a PCI card. As depicted, the PCI includes a terminal block and a set of fuse holders integrated therewith. It will be appreciated that the terminal block can be a process control terminal block that is coupled with control elements (e.g., control valves, heating elements, pumps, etc.) that can be controlled by signals applied to the control block. For example, in a pasteurization process, the terminal block can have an output coupled with a divert/flow valve (e.g., divert flow valve 106 of FIG. 1). In such an embodiment, once a determination is made that the product undergoing pasteurization has met the legal requirements to be considered pasteurized, the PCI card can apply a signal to a terminal of the terminal block to cause the divert/flow valve to open up to allow the product to exit the pasteurization process. In some embodiments, the PCI card can be configured to operate the terminal block in a low voltage (e.g., 40 volts) DC mode. In such embodiments, the control elements would also need to be operated via a correspondingly low voltage. In other embodiments, the PCI card can operate at a typical voltage (e.g., 120 v/240 v).

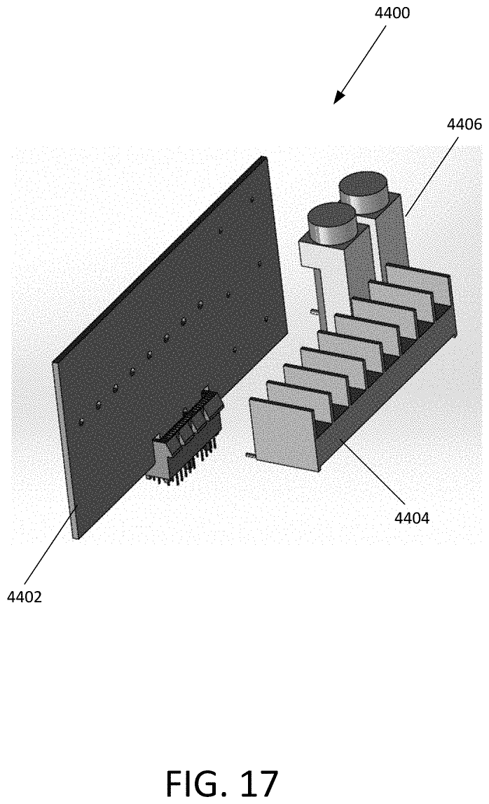

FIG. 17 is an illustrative exploded view of an example pasteurization control interface (PCI) card assembly 1700, in accordance with various embodiments of the present disclosure. As can be seen, the PCI card assembly includes a circuit board 1702, a terminal block 17134, and a set of fuses. Each of these components has been described in detail already and therefore will not be described further in reference to this figure.

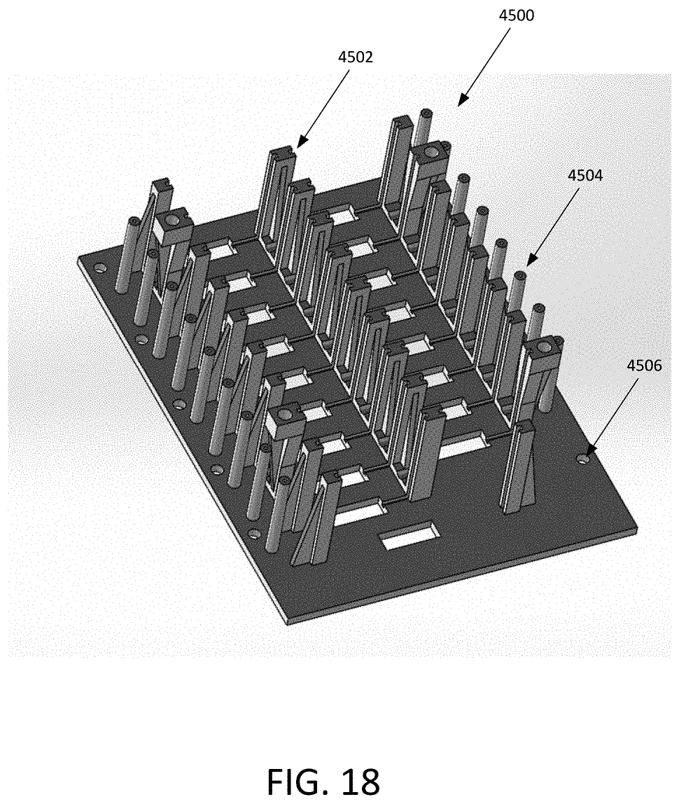

FIG. 18 is an illustrative view of an example card guide plate 1800, in accordance with various embodiments of the present disclosure. Card guide plate 1800 can be configured to guide cards into proper placement with respect to the backplane circuit board utilized in some embodiments. As can be seen, card guide plate includes guide elements 1802, also referred to herein as card sliding holder, in which the cards can slide when being installed into the backplane circuit board. In some embodiments, guide elements 1802 can include a latch mechanism that engages a corresponding latch slot of a card being installed. Such a latch mechanism can engage the latch slot to help retain an installed card once it is installed and to indicate adequate installation of the card to help ensure greater than necessary force is not applied during card installation. As can be seen, there are pass-through elements of the card guide plate that allow access to sockets of the backplane circuit board. The card guide plate can be installed in conjunction with a backplane circuit board via installation holes 1806.

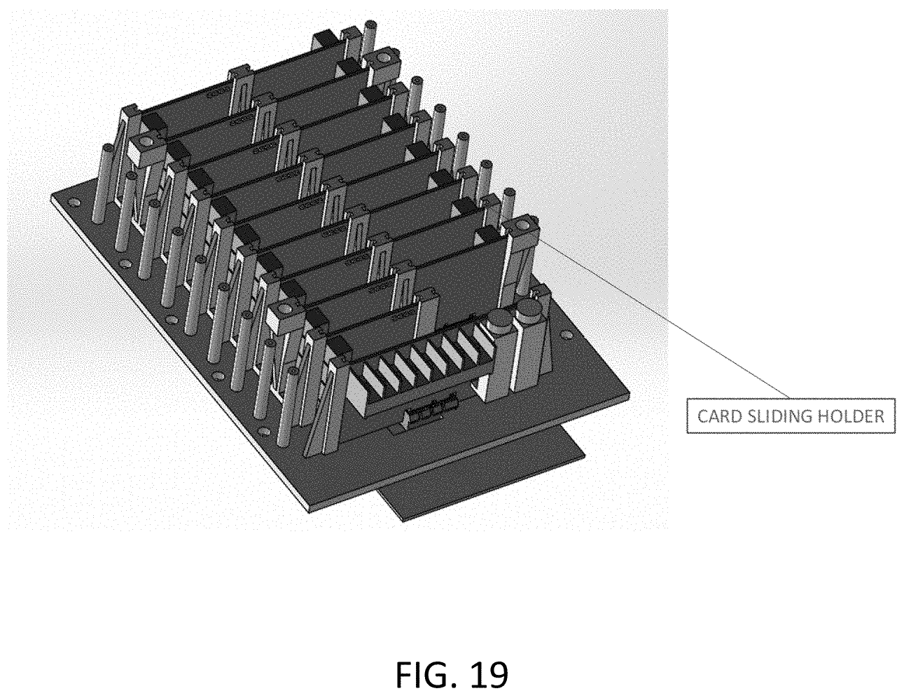

FIG. 19 is an illustrative view of an example circuit board assembly utilizing card guide plate 1800, in accordance with various embodiments of the present disclosure.

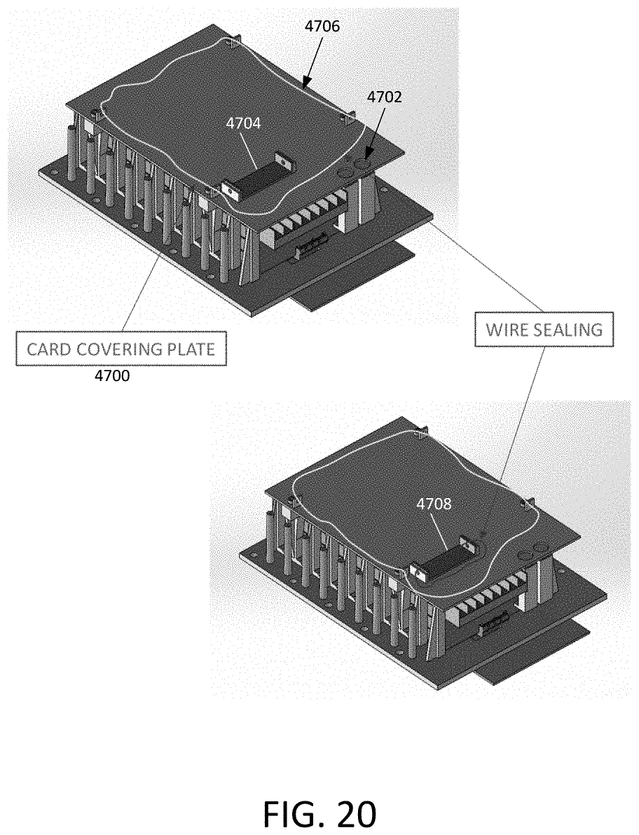

FIG. 20 is an illustrative view of an example card covering plate 2000 and a programming covering plate 2004 having respective wire seals 2006 and 2008, in accordance with various embodiments of the present disclosure. In some embodiments it may be necessary to seal certain components of the paperless recording control system from outside tampering, or at least to indicate whether the integrity of the paperless recording control system has been breached. An example of such an embodiment is reflected in a pasteurization scenario where changes to the system may be limited by regulators (e.g., FDA) and any tampering with the system could compromise the pasteurization process. In such embodiments, card covering plate 2000 and a corresponding wire seal 2006 can be installed to prevent manipulation of the cards installed in the paperless recording control system. In some embodiments, not depicted, the card covering plate can be completely solid to completely cover the entirety of the circuit board assembly. In other embodiments, such as the depicted one, it may be desirable to have access to certain components that may be able to be accessed without completely compromising the functionality and integrity of the paperless recording control system. For example, as mentioned above, in some instances there can be a set of fuses integrated with the circuit board assembly. To cover these fuses and require regulatory sign-off for replacement of these fuses may not be necessary and as such an access port 2002 for each fuse can be utilized for replacing these fuses.

In addition, it may be desirable to enable certain changes to the programming of the system (e.g., via programming switches), so long as the integrity of the cards is maintained. In such instances a separate programming covering plate 2004 can be utilized to enable access to a programming port of the paperless recording control system. In such embodiments, it may still be desirable to seal the programming covering plate to indicate unauthorized access to the programming port. In such embodiments a separate seal can be utilized to indicate the integrity of the programming port.

As mentioned previously, in some embodiments the paperless recording control system can include removable storage (e.g., thumb drive, SD card, etc.). In such embodiments, a separate cover plate and associated seal can be utilized in some circumstances to ensure the integrity of the removable storage.

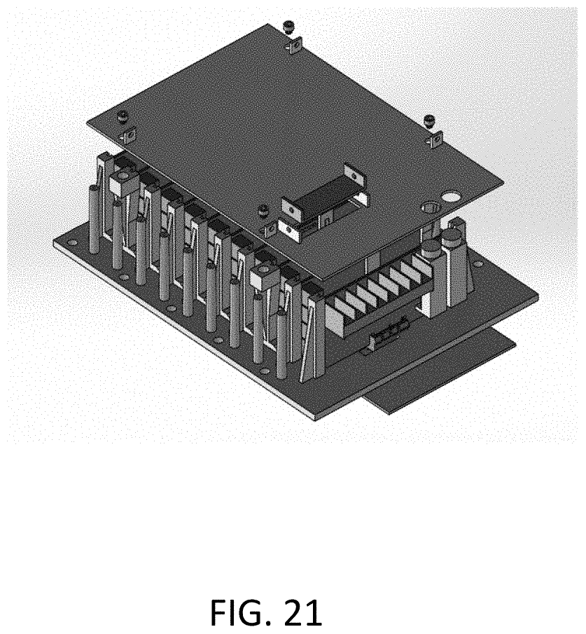

FIG. 21 is an illustrative component view of card covering plate 2000 and programming covering plate 2004, in accordance with various embodiments of the present disclosure. These components have been thoroughly described above and therefore will not be further described in reference to FIG. 21.

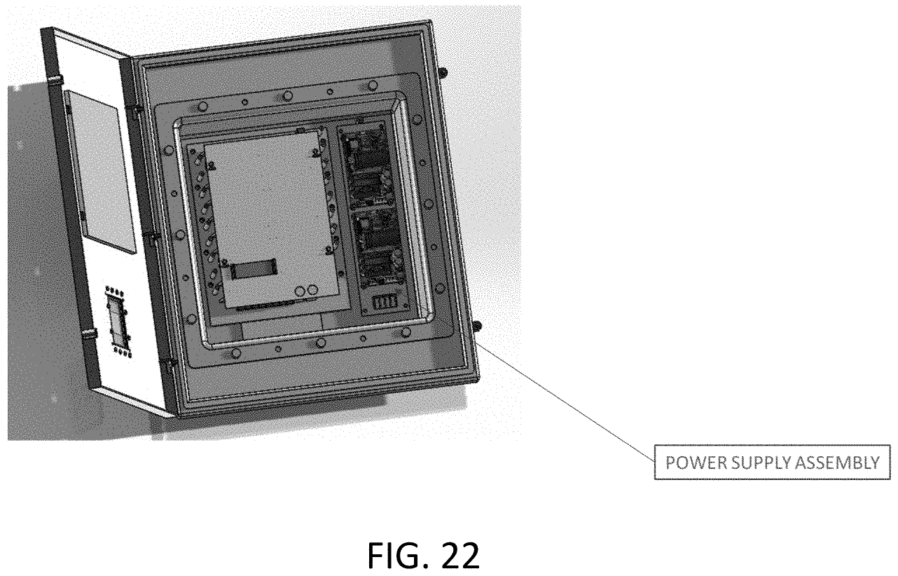

FIG. 22 is an internal view of an enclosure having a paperless recording control system with card covering plate 2000 and programming covering plate 2004, along with an associated power supply assembly 2202, in accordance with various embodiments of the present disclosure. As depicted there can be two distinct power supplies, one for the HMI system (e.g., HMI system 208 of FIG. 2) and one for the control system (e.g., control system 202 of FIG. 2). Having two distinct power supplies enables the HMI system and the control system to be decoupled from a power supply perspective to help ensure that the control system is a hard real-time system. A third power supply can also be utilized for the reference system to ensure the reference system is also decoupled from the other systems and can operate independently in isolation, however, in the depicted embodiment, such a power supply is not depicted, or at least not distinguishable.

In addition, in some embodiments, the power supplies are AC providing voltage of, for example, 120 v or 240 v. In other embodiments, the power supplies can be supplied with DC current, or can be configured to convert AC current applied to DC current for use by the paperless recording control system. Such a DC embodiment can be utilized, for example, in a low voltage (e.g., 40 v) implementation in which the entire paperless recording control system is operated in DC.

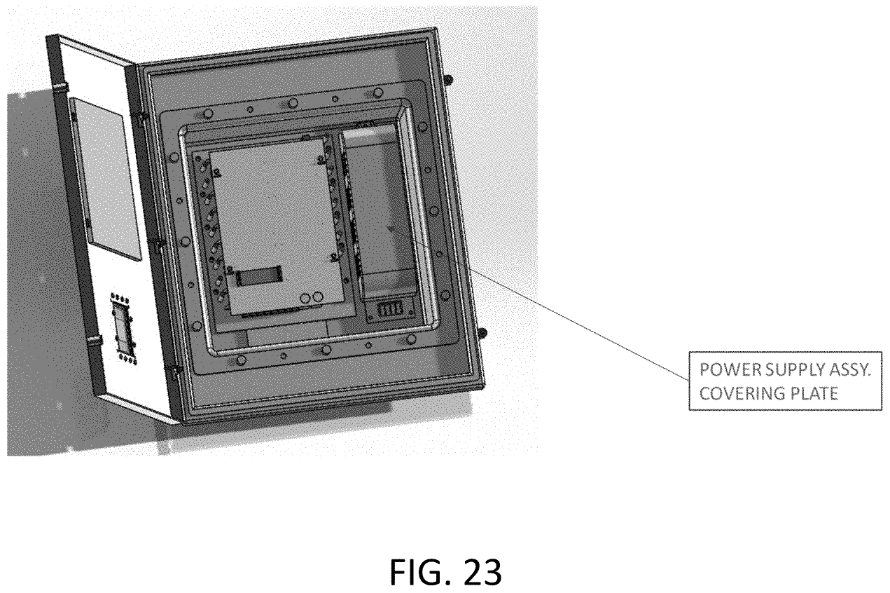

FIG. 23 is another internal view of the enclosure depicted in FIG. 21 having a covering plate installed over the power supplies discussed above, in accordance with various embodiments of the present disclosure. The power supply cover plate can help to prevent accidental shock that can be caused by the power supplies/

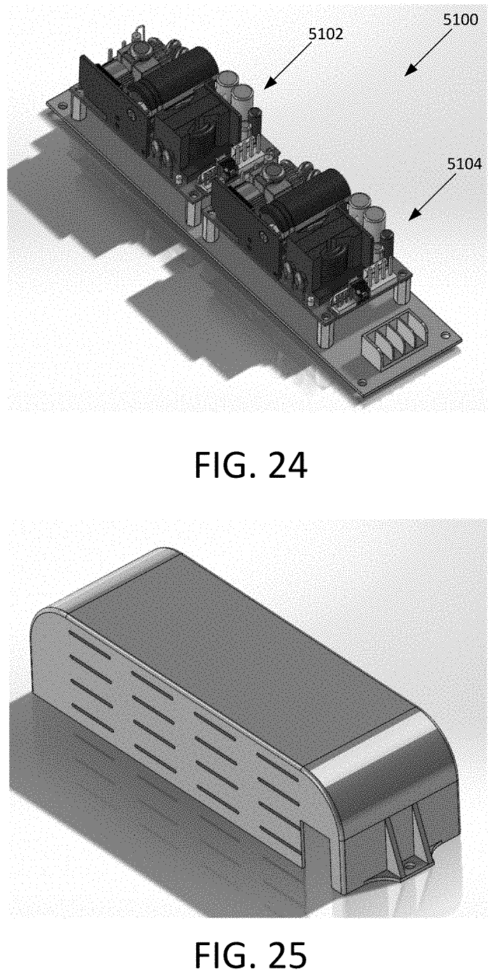

FIG. 24 is a view of an example power supply assembly 2400, in accordance with various embodiments of the present disclosure. As depicted, power supply 2400 includes a first power supply 2402 for providing power to the control system and a second power supply 2404 for supplying power to the HMI system.

FIG. 25 is a view of an example power supply assembly covering plate, in accordance with various embodiments of the present disclosure.

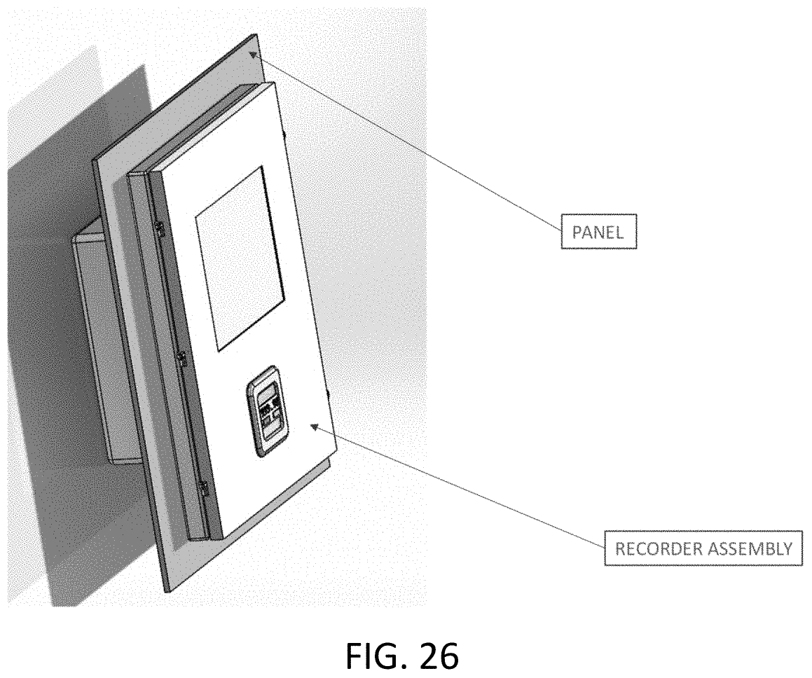

FIG. 26 is a view of an enclosure for a paperless recording assembly integrated with a panel, in accordance with various embodiments of the present disclosure. In embodiments, the enclosure can be configured for either mounting in a panel (e.g., in place of existing paper recorders) or wall mounting the enclosure (not depicted) without the need for a panel.

Computer storage media, or any other media described herein, includes volatile and nonvolatile, removable and non-removable media implemented in any method or technology for storage of information such as computer-readable instructions, data structures, program modules, software, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by a computing device. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory includes computer storage media in the form of volatile and/or nonvolatile memory. Memory can include instructions, which, when executed by a processor are configured to cause a computing device to perform any of the operations described herein, in reference to the above discussed figures, or to implement any program modules or components described herein. The memory may be removable, non-removable, or a combination thereof. Illustrative hardware devices include solid-state memory, hard drives, optical-disc drives, etc. Embodiments presented herein have been described in relation to particular embodiments which are intended in all respects to be illustrative rather than restrictive. Alternative embodiments will become apparent to those of ordinary skill in the art to which the present disclosure pertains without departing from its scope.

From the foregoing, it will be seen that this disclosure in one well adapted to attain all the ends and objects hereinabove set forth together with other advantages which are obvious and which are inherent to the structure.

It will be understood that certain features and sub-combinations are of utility and may be employed without reference to other features or sub-combinations. This is contemplated by and is within the scope of the claims.

In the preceding detailed description, it is to be understood that other embodiments may be utilized and structural or logical changes may be made without departing from the scope of the present disclosure. Therefore, the preceding detailed description is not to be taken in a limiting sense, and the scope of embodiments is defined by the appended claims and their equivalents.

Various aspects of the illustrative embodiments have been described using terms commonly employed by those skilled in the art to convey the substance of their work to others skilled in the art. However, it will be apparent to those skilled in the art that alternate embodiments may be practiced with only some of the described aspects. For purposes of explanation, specific numbers, materials, and configurations are set forth in order to provide a thorough understanding of the illustrative embodiments. However, it will be apparent to one skilled in the art that alternate embodiments may be practiced without the specific details. In other instances, well-known features have been omitted or simplified in order not to obscure the illustrative embodiments.

Various operations have been described as multiple discrete operations, in turn, in a manner that is most helpful in understanding the illustrative embodiments; however, the order of description should not be construed as to imply that these operations are necessarily order dependent. In particular, these operations need not be performed in the order of presentation. Further, descriptions of operations as separate operations should not be construed as requiring that the operations be necessarily performed independently and/or by separate entities. Descriptions of entities and/or modules as separate modules should likewise not be construed as requiring that the modules be separate and/or perform separate operations. In various embodiments, illustrated and/or described operations, entities, data, and/or modules may be merged, broken into further sub-parts, and/or omitted.

The phrase “in one embodiment” or “in an embodiment” is used repeatedly. The phrase generally does not refer to the same embodiment; however, it may. The terms “comprising,” “having,” and “including” are synonymous, unless the context dictates otherwise. The phrase “A/B” means “A or B.” The phrase “A and/or B” means “(A), (B), or (A and B).” The phrase “at least one of A, B and C” means “(A), (B), (C), (A and B), (A and C), (B and C) or (A, B and C).”

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention. Accordingly, the invention should not be limited except as by the appended claims.

1. A data recording system, comprising:
a temperature sensor connected to a vessel structured to hold a food substance, the temperature sensor structured to determine an instant temperature of the food substance;
a control signal generator to produce a control signal; and
a processor configured to:
receive the instant temperature from the temperature sensor,
record a series of the instant temperatures in an internal memory each along with a respective time at which the instant temperature was recorded;
send a copy of the series of recorded time and temperatures to a remote data collection facility; and
based on the series of recorded time and temperatures, cause the control signal generator to generate the control signal.

2. The data recording system according to claim 1, wherein the temperature sensor is a digital reference temperature sensor.

3. The data recording system according to claim 1, wherein the series of recorded time and temperatures is stored in the internal memory in an encrypted format.

4. The data recording system according to claim 1, wherein the internal memory includes a permanent internal media portion and a removable media portion.

5. The data recording system according to claim 1, wherein the temperature sensor, the control signal generator, and the processor are physically located in a same housing, and wherein the temperature sensor is coupled to a first power supply in the housing and the processor is coupled to a second power supply in the housing.

6. The data recording system according to claim 1, further comprising a human machine interface including an output for providing visible information and an input for accepting user commands.

7. The data recording system according to claim 1, further comprising a pressure sensor, and wherein the processor is configured to:
receive an instant pressure reading from the pressure sensor; and
record a series of the instant pressure readings in the internal memory each along with a respective time at which the instant pressure reading was recorded.

8. A pasteurization system comprising:
a flow sensor connected to a pasteurization tube to determine a flow rate of a food substance to be pasteurized;
a temperature sensor connected to the pasteurization tube, the temperature sensor structured to determine an instant temperature of the food substance;
a control signal generator to produce a control signal; and
a processor configured to:
receive an instant flow rate of the food substance in the pasteurization tube,
receive the instant temperature from the temperature sensor,
record a series of the instant temperatures in an internal memory each along with a respective time at which the instant temperature was recorded;
send a copy of the series of recorded time and temperatures to a remote data collection facility; and
based on the series of recorded time and temperatures, cause the control signal generator generate the control signal to modify the flow rate of the food in the pasteurization tube.

9. The pasteurization system according to claim 8, wherein the temperature sensor is a digital reference temperature sensor.

10. The pasteurization system according to claim 8, wherein the series of recorded time, temperatures, and pressures is stored in the internal memory in an encrypted format.

11. The pasteurization system according to claim 8, wherein the internal memory includes a permanent internal media portion and a removable media portion.

12. The pasteurization system according to claim 8, wherein the temperature sensor, the control signal generator, and the processor are physically located in a same housing, and wherein the temperature sensor is coupled to a first power supply in the housing and the processor is coupled to a second power supply in the housing.

13. The pasteurization system according to claim 8, further comprising a human machine interface including an output for providing visible information and an input for accepting user commands.

14. A method in a pasteurization system for pasteurizing a food substance, the method comprising:
receiving an instant temperature from a temperature sensor positioned to measure an instant temperature of the food substance;
recording a series of the instant temperatures in an internal memory each along with a respective time at which the instant temperature was recorded;
sending a copy of the series of recorded time and temperatures to a remote data collection facility; and
based on at least the series of recorded time and temperatures, generating a control signal that causes the pasteurization system to modify its process for pasteurizing the food substance.

15. The method according to claim 14, wherein the temperature sensor is a digital reference temperature sensor.

16. The method according to claim 14, wherein recording a series of the instant temperatures in an internal memory comprises recording the series in an encrypted format.

17. The method according to claim 14, wherein the internal memory includes a permanent internal media portion and a removable media portion.

18. The method according to claim 14, wherein the temperature sensor, the control signal generator, and the processor are physically located in a same housing, and wherein the temperature sensor is coupled to a first power supply in the housing and the processor is coupled to a second power supply in the housing.

19. The method according to claim 14, further comprising accepting one or more user commands at a human machine interface.

20. The method according to claim 14, further comprising:
receiving an instant pressure reading from a pressure sensor; and
recording a series of the instant pressure readings in the internal memory each along with a respective time at which the instant pressure reading was recorded.
